(19) United States
(12) Patent Application Publication
(10) Pub. No.: US 2008/0147106 A1
(43) Pub. Date: Jun. 19, 2008

(54) Surgical instrument

(75) Inventors: Volker Mohr, Nutzen (DE); Walter Jozat, Bad Bramstedt (DE); Andreas Jaudszims, Henstedt-Ulzburg (DE)

Correspondence Address: Horst M. Kasper, 13 Forest Drive, Warren, NJ, 07059, US

(73) Assignee: SORING GMBH

(21) Appl. No.: 12/002,554

(22) Filed: Dec. 18, 2007

(30) Foreign Application Priority Data
Dec. 18, 2006 (DE) 10 2006 059 728.1

(51) Int. Cl. A61B 17/3201

(52) U.S. Cl. 606/169

(57) ABSTRACT

The surgical instrument comprises a generator and control unit, a utility line connected to the generator and control unit, a converter connected to the utility line for converting electrical energy into ultrasound, a casing attached to the converter and having an outer cylindrical section and having a central axial bore hole; a slider block slidable over the cylindrical section, a fixed handle part attached to the casing at a first end, a movable handle part attached to the slider block at a first end and joining the fixed handle part, a sonotrode attached to the converter and running through the central bore hole of the casing, a covering sleeve covering the sonotrode over a front region, a push rod attached to the slider block and running inside the covering sleeve, a clamping jaw hinged to the sonotrode and connected to the push rod for pivoting the clamping jaw hinged to the sonotrode.

### BACKGROUND OF THE INVENTION

(1) Field of the Invention

A surgical instrument, in particular ultrasonic scissors employed in connection with laparoscopic operations, which ultrasound scissors are employed for cutting and coagulating, are the subject matter of the present invention. Such scissors are also usable, of course, for open surgical operations in a shortened embodiment.

(2) Description of Related Art Including Information Disclosed Under 37 CFR 1.97 and 1.98

Different apparatuses have become known from the state of the art. For example a surgical ultrasound instrument for cutting, coagulating, and gripping is described in the international patent application number WO 94/16631. This instrument is intended for the use in connection with laparoscopic operations. Construction and handling imitate that of standard scissors, that is by moving of two levers actuated by the fingers and the thumb of the operator there is a clamping lever swivelable against a fixed position blade and depending on the size of the press on pressure, the tissue disposed between clamping jaw and blade can be cut or squeezed. In this case the blade is connected to a converter, wherein the electrical energy fed in from a generator through a connection cable is transformed into ultrasound oscillations in the converter. A coagulation, that is a welding of blood vessels is made possible with the aid of these ultrasound oscillations.

It is important for an interference free functioning of the instrument, that the blade formed as a sonotrode is connected clean and solid to the converter. It is necessary for this purpose that the sonotrode is screwed into an attachment thread of the converter. The converter and the sonotrode are then introduced into a corresponding bore hole of the scissors and are secured against sliding out by a clamping lever. This method is expensive and intensive in materials since the scissors have to be thrown out after each operation because the scissors cannot be treated in autoclave.

Ultrasound scissors are also known from the German Gebrauchsmuster 20118698, where the ultrasound scissors can be subdivided for the purpose of making them germ free in an autoclave by taking out the push rod out of clamping jaw and sliding block after withdrawal of a covering sleeve, the handle is unhooked at the converter receiver, and can be withdrawn together with the sliding block from the guide tube. After the re-mounting the push rod for actuating the movable clamping jaw is fixed in a groove of the guide tube by the slid on covering sleeve. The German Gebrauchsmuster No. 201 18698 describes ultrasound scissors to be employed for cutting and coagulating. The handle is formed as a single piece made out of spring steel, wherein one end of the handle is hingedly attached at the converter receiver and the second end is attached at the sliding block.

It is a deficiency of the two known solutions that the surgeon, during an operation in a change from a dissection mode or from a cutting or, respectively, coagulation mode for flushing or suctioning off, has to take the ultrasound scissors out of his hand and has to receive a flushing or suctioning apparatus. Possibly it is necessary to place in addition an additional trocar.

An ultrasonic surgical device is taught in United States Patent Application Publication US 2005/0049546 A1 published Mar. 3, 2005 to Messerly et al. The device is intended for endoscopic operations and can serve to cut, to coagulate, and to dissect.

The German Printed Patent Publication DE 40 32 471 A1 to Eberhard Roos teaches an electro surgical device. The reference teaches that a suitable RF-current can be applied to the coagulation electrodes (22, 23).

The German Petit Patent Gebrauchsmuster DE 203 03 777 U1 2004/01.22 to Söring GmbH teaches a medical treatment apparatus. A connection (12, 13) is furnished for accepting a suction hose (14).

The Translation into German of the European Patent DE 694 32 741 T2 2004.03.25 to Davis et al. teaches a coagulation/cut clamp system for surgical ultrasonic instruments. The ultrasound instrument 10 comprises a casing or handle piece 14, an ultrasound drive and converter, preferably a piezo ceramic converter for converting the electrical signal.

The Patent Abstracts of Japan, Publication number 08275951 A published 22.10.96 to Okada Mitsumasa et al. teaches an ultrasonic dissecting and coagulating device. A dissecting part 7a and a coagulating part 7b are freely selected by operating the operating ring by an operator.

### BRIEF SUMMARY OF THE INVENTION

### PURPOSES OF THE INVENTION

It is an object of the present invention to furnish a surgical combination instrument in the shape of multiply again usable, autoclavable ultrasound scissors, which combination instrument offers both the dissection, that is the fragmentation of tissue, as well as also a scissors function for cutting and coagulating.

These and other objects and advantages of the present invention will become evident from the description which follows.

### BRIEF DESCRIPTION OF THE INVENTION

This object is obtained by forming this instrument as a combination instrument, wherein the combination instrument combines both the possibility of the dissection as well as also of the cutting and of the coagulation of tissue.

The surgical instrument is represented in particular by ultrasound scissors employed in laparoscopic operations, which scissors are employed for cutting of tissue and for coagulation of vessels, wherein this instrument is formed as a combination instrument which unites both the possibility of dissection and also of cutting and of coagulation of tissue.

The surgical instrument comprises a generator and control unit, a utility line connected to the generator and control unit, a converter connected to the utility line for converting electrical energy into ultrasound, a casing attached to the converter and having an outer cylindrical section and having a central axial bore hole; a slider block slidable over the cylindrical section, a fixed handle part attached to the casing at a first end, a movable handle part attached to the slider block at a first end and joining the fixed handle part, a sonotrode attached to the converter and running through the central bore hole of the casing, a covering sleeve covering the sonotrode over a front region, a push rod attached to the slider block and running inside the covering sleeve, a clamping jaw hinged to the sonotrode and connected to the push rod for pivoting the clamping jaw hinged to the sonotrode.

The present instrument is a combination of two methods employed in the field of ultrasound surgery. One aspect would be the ultrasound cutting or, respectively, ultrasound coagulating which usually is performed at a frequency of 55 kHz and with sonotrodes made of full material. A second aspect would be the ultrasound dissection, where cavitation forces are employed with a tubular sonotrode in order to present selectively tissues and to prepare the tissues in a free form. The dissection occurs mostly at a frequency of 25 or 35 kHz as a working frequency. An instrument is provided which combines cutting, coagulating and dissecting. A tubular sonotrode can perform a dissection and a frequency of 25 or 35 kHz or also a cut or coagulation at a frequency of 55 kHz.

Lines for flushing and for suctioning can be disposed in and/or at the casing of the converter receiver. A connection for connecting to a suction pump can be furnished at the free end of the suctioning line. A second connection for connecting to the flushing pump can be furnished at the free end of the flushing line. The sonotrode can be equipped with an axial channel. The axial channel of the sonotrode can be employed as a suction and/or as a flushing channel. The flushing of the application location can be performed by a line disposed inside the sonotrode, or the flushing of the application location is performed by a line disposed parallel to the sonotrode.

The control of the suction and flushing pumps can be triggered by push keys. The push keys can be arranged in the rigid part of the handle or the push keys can be arranged in the movable part of the handle. The control of the suction and flushing pumps can be triggered by a foot switch. The sonotrode and the covering sleeve can be formed exchangeable. The rigid and the movable part of the handle can be connected by a spring made of stainless steel. The clamping face of the clamping jaw can be furnished with an anti-attachment coating, for example made out of poly tetra fluoro ethylene.

A converter receiver can support the converter. A flushing line can run substantially parallel to the converter receiver and have at one end a connecter for a flushing line leading to a flushing pump. A suctioning line can run substantially parallel to the converter receiver and have at one end a connecter for a suctioning line leading to a suction pump. An axial channel can be disposed around the center axis of the sonotrode and inside the sonotrode, wherein the axial channel of the sonotrode is employed as a suction and/or flushing channel.

The flushing of the application location can be performed by a line disposed inside the sonotrode. The flushing of the application location can be performed by a line disposed parallel to the sonotrode. A push key can be furnished for triggering a control of the suction pump or of the flushing pump. The push key can be arranged in the rigid part of the handle or in the movable part of the handle. A foot switch can be connected to the generator or control unit for triggering a control of the suction pump or flushing pump.

The sonotrode and the covering sleeve can be formed exchangeable. A clamping face of the clamping jaw can be furnished with an anti-attachment coating. The fixed part of the handle and the movable part of the handle can be connected to each other by a spring made of stainless steel. The fixed part of the handle can be disposed relatively remote to a tip of the sonotrode, and the movable part of the handle can be disposed relatively close to the tip of the sonotrode,

A clamping screw can be supported by the casing, disposed between the fixed part of the handle and the movable part of the handle and engaging the sonotrode for securing the sonotrode against unintentional rotation, wherein the clamping screw extends in a radial direction relative to an axis of the sonotrode, wherein the clamping screw has a grippable head for manual operation and tightening.

An outer threaded end of the casing can be disposed adjoining the cylindrical section of the casing. A swivel nut can have an inner thread matching the outer threaded end of the casing and engaging the outer threaded end and having a perforated ring on an end disposed remote to the casing, wherein said perforated ring has an inner diameter larger than an outer diameter of the covering sleeve, wherein the swivel nut has a length longer than a length of the sliding block, and wherein the swivel nut has an outer diameter larger than an outer diameter of the sliding block, and wherein the swivel nut serves as a stop for the sliding block.

A method of ultrasonic surgery comprises the steps of attaching a fixed end of a handle to a casing, attaching a movable end of a handle to a sliding block sliding over a section of the handle, placing a sonotrode through a central axial bore hole of the casing, attaching the sonotrode to a converter, connecting a push rod to the sliding block, surrounding the push rod and the sonotrode with a covering sleeve, and screwing a swivel nut onto a threaded end of the casing for furnishing a stop to the sliding block. Switching can be performed between a cutting and coagulating operation and a dissecting operation. A push key can be pressed for switching between the cutting and coagulating operation and the dissecting operation.

The general development of the costs in health care establishments requires that also in the region of surgery the one-way apparatuses as far as possible are replaced by multiply usable apparatuses. This means for the use of ultrasound scissors that these scissors have to be formed such that feed-in line, converter and sonotrode as well as the construction unit “the scissors” are cleaned and sterilized in an autoclave. For this purpose it is required that the instrument can be easily disassembled, that means without using an additional tool and can be assembled again also easily after the treatment in the autoclave. Particular helpful is the combination of several individual apparatuses to a combination apparatus, where different operations can be performed with the combination apparatus without that an instrument is to be laid out of hand and another instrument has to be picked up.

The subject matter of the present invention is to be described in more detail by way of an embodiment example.

The novel features which are considered as characteristic for the invention are set forth in the appended claims. The invention itself, however, both as to its construction and its method of operation, together with additional objects and advantages thereof, will be best understood from the following description of specific embodiments when read in connection with the accompanying drawing.

### BRIEF DESCRIPTION OF THE DRAWING

In the accompanying drawing, in which are shown several of the various possible embodiments of the present invention:

FIG. 1 an overall view of an ultrasound instrument with generator, scissors, converter, and connections for flushing lines and suctioning lines;

FIG. 2 a partial view from the top onto the converter receiver and the flush lines and suctioning lines;

FIG. 3 a sectional view through a sonotrode assembly with covering sleeve.

### DETAILED DESCRIPTION OF THE INVENTION

An overall arrangement comprising a generator and control unit (1), a feed line (2), converter receiver (3) and scissors is shown in FIG. 1. The support (28) and the connection bridge (27) for the flushing line (9) and the suctioning line (10) disposed in the connection bridge (27) are attached on the casing of the converter receiver (3). The connections (4) and (5) are furnished in the shape of quick couplings at the free ends of the flushing or, respectively, suctioning lines, wherein the connection hoses to the suction pump (7) or, respectively, to the flushing pump (6) not shown here can be plugged into. The flushing pump and the suction pump can be controlled through push keys (29, 30) disposed in the gripping region of the scissors for flushing pump (6) and suction pump (7). The exact position of the push keys (29, 30) is fixed only after detailed ergonomic investigations, for example it is also conceivable to place the push keys in the movable part of the handle. The cable connection to the generator and control unit (1) is performed by the feed line (2). This feed line (2), which is plugged into the connection bush (12), receives also the cables necessary for the voltage transmission to the converter. The flushing lines and the suctioning lines can be connected to the corresponding connectors at the hand piece and pumps if needed. The suctioned off liquids can be collected in a commercial container.

The cable (24) can be connected to the bush (26) of the generator and control unit (1) in addition to the hand actuation at the generator and control unit (1), wherein the cable (24) connects a foot switch (25) to the generator and control unit (1).

The sonotrode (20) protrudes out of the distal end of the covering sleeve (15) and is with its rearward end screwed into the converter. The sonotrode (20) passes through the center of the casing (17). The sonotrode is secured with a clamping screw (11) against an unintentional rotation. The clamping screw (11) is movable in a plane disposed perpendicular to a central axis of the casing (17). The clamping screw (11) presses against the outer surface of the sonotrode (20) and thereby maintains a radial position and/or an axial position of the sonotrode (20). Preferably the clamping screw moves in a radial direction relative to an axis of the casing (17).

The sonotrode represents a peculiarity. The sonotrode is furnished with an axial bore hole through which the wound drainings and/or flushing liquid can be fed in or, respectively, can be suctioned off. Flushing liquid fed in through the line (9) is guided to the tip of the sonotrode through a channel furnished within the sonotrode. Liquid and tissue parts are also suctioned off through a line disposed inside the sonotrode.

Alternatively, the flushing liquid can also flow between the outside of the sonotrode (20) and the inside of the covering sleeve (15) and this way pass to the tip of the sonotrode (20). These lines (9,10) of course can also be disposed outside and parallel to the sonotrode.

Since differently long sonotrodes and guide sleeves are required for the different operational tasks, there is provided a corresponding change at the hand piece. For this purpose the swivel nut (16) is screwed off and the transfer rod not illustrated here to the clamping jaw (18) is unhooked. Thereupon the covering sleeve (15) with the clamping jaw (18) attached thereto can be pulled off. After the removal of the covering sleeve (15), then the sonotrode can be screwed out of the converter after the release of the clamping screw (11) and can be exchanged against a longer or shorter sonotrode.

The tubular sonotrode (20) also serves for suctioning off the disintegrated tissue and can also transport flushing liquid to the tissue with an interior disposed flushing hose.

The handle (14) comprises two plastic parts, which are connected to each other with a spring steel sheet metal strip. This construction is more friendly to the hand as compared to a metal only handle since the plastic can be easier formed as compared with the spring steel.

The rigid part of the handle (14) is solidly connected to the casing (17) of the hand piece. The handle (14) comprises a fixed arm and a shiftable arm. The fixed arm of the handle is attached to the casing in the area between the converter receiver (3) and the clamping screw (11). The fixed arm of the handle (14) is furnished with push keys (29, 30) for controlling the suction and the flushing flow. The casing (17) extends from the converter receiver (3) to the covering sleeve (15). There can also be push keys (29, 30) for transitioning from a cutting and coagulating operation to a dissecting operation and vice versa.

The front region of the casing (17) ends with a thread for the swivel nut (16) and is formed cylindrically over a limited length. An end of the casing (17) near the covering sleeve carries a thread to be engaged by the swivel nut (16). Adjoining the thread of the casing (17) is a cylindrical section of the casing (17). The sliding block (13) slides over the cylindrical section of the casing (17) and preferably has the shape of a ring. The swivel nut (16) delimits the path of the sliding block (13) toward the tip of the sonotrode (20). The swivel nut (16) preferably exhibits a front ring covering the thread of the casing (17) and having an diameter for allowing the covering sleeve (15) to pass through the front ring. The outer diameter of the swivel nut (16) preferably larger than the outer diameter of the sliding block (13). The axial length of the swivel nut (16) is preferably larger than the axial length of the sliding block (13).

The sliding block (13) slides over the cylindrical region of the casing (17) and is axially transposable by way of the free arm of the handle (14). In order to assure problem free working with the scissors, the free part of the handle (14) is connected hingedly to the sliding block (13) with the connection (23).

The force transfer from the handle (14) through the sliding block (13) to the to the clamping jaw (18) is performed in a known fashion through push rod not illustrated here should bear the push rod engages the clamping jaw (18) supported in the rotation point (22) from the side.

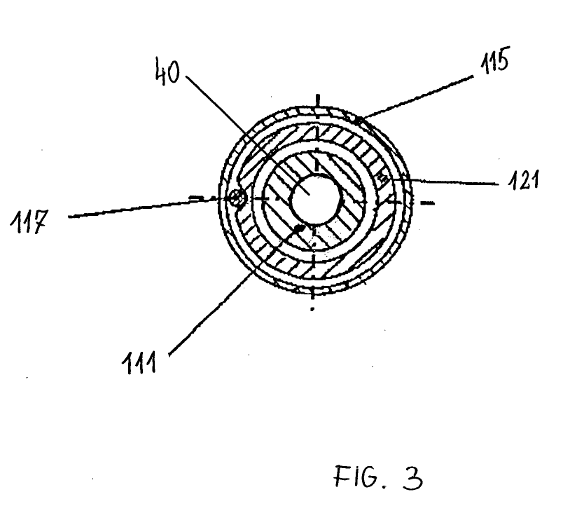

It can be recognized from FIG. 3 that the push rod (117) is supported in an axially running groove like recess of the guide tube (121). The push rod (117) is fixed in the recess by the covering sleeve (115) slid onto the guide tube (121). A small intermediate space was drawn between the guide tube (121) and the covering sleeve (115) for the purpose of better understanding the section of FIG. 3. In the embodiment in reality the covering sleeve (115) rests sealingly at the guide tube (121). The groove like recess for the push rod (117) is of such depth that the push rod (117) can be easily moved in axial direction while the covering sleeve (115) is slid on. The suction line (40) is disposed inside of the sonotrode (111). A flushing line can be disposed outside of the sonotrode (111) and inside of the covering sleeve (115) and in particular between the sonotrode (111) and the guide tube (121).

The handle (14) comprises a stainless spring steel such that pressing together of the two gripping parts is opposed by the spring force. The push rod is moved in the direction toward the converter and therewith the clamping jaw (18) is pressed against the sonotrode (20) based on the pressing together. Depending on the intensity and duration of the pressing together, the surgeon can cut with these scissors or also only weld tissue while the ultrasound energy is applied.

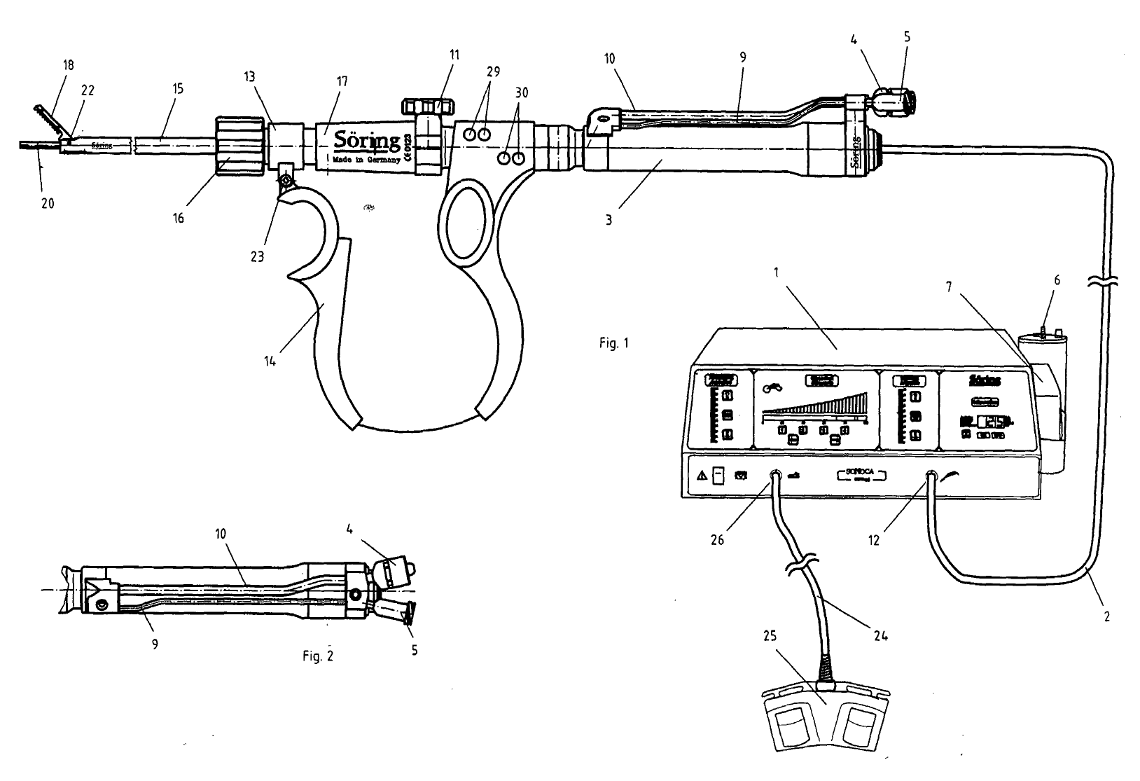

FIG. 2 shows a part view from the top onto the converter receiver and the flushing and suctioning lines. Here it can be clearly recognized that the flushing line (9) and the suctioning line (10) are disposed lying next to each other in the casing of the converter receiver (3). The lines (8,9) are supported by the support (28) placed at the converter receiver in the region of the connections (4,5). The connections (4,5) are situated outside of this support (28) in order to not interfere with this quick change of the lines. The two lines (9,10) end in the connection bridge (27), wherein the connection bridge (27) is sealingly attached on the converter receiver (3). Channels in the interior of the apparatus lead from the connection bridge to the opening in the sonotrode or, respectively, to the covering sleeve (15).

It has been observed during operation that blood and tissue residues are attached at the clamping jaws (18) of ultrasound scissors, which blood and tissue residues can only be removed with very much patience. For this reason, the clamping jaw of the ultrasound scissors can be equipped with an exchangeable pressure plate. The pressure plate is simply produceable and a relatively price favorable part, which can be exchanged either after conclusion of an operation or also in between.

It will be understood that each of the elements described above, or two or more together, may also find a useful application in other types of ultrasonic treatment system configurations and surgical processing procedures differing from the types described above.

While the invention has been illustrated and described as embodied in the context of an ultrasonic instrument for cutting, coagulating and dissecting, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

### LIST OF REFERENCE CHARACTERS

1 generator and control unit
2 feed line
3 converter receiver
4 connection for suction line
5 connection for flushing line
6 flushing pump
7 suctioning pump
9 flushing line
10 suctioning line
11 clamping screw
12 connection bush
13 sliding block
14 handle
15 covering sleeve
16 swivel nut
17 casing
18 clamping jaw
20 sonotrode
22 rotation point
23 connection
24 cable
25 foot switch
26 bush
27 connection bridge
28 support
29 push key for flushing pump
30 push key for suction pump
40 suctioning line
111 sonotrode
115 covering sleeve
117 push rod
121 guide tube

1. A surgical instrument, in particular ultrasound scissors employed in laparoscopic operations, which scissors are employed for cutting of tissue and for coagulation of vessels, wherein this instrument is formed as a combination instrument which unites both the possibility of dissection and also of cutting and of coagulation of tissue.

2. A surgical instrument comprising
a generator and control unit;
a utility line connected to the generator and control unit;
a converter connected to the utility line for converting electrical energy into ultrasound;
a casing attached to the converter and having an outer cylindrical section and having a central axial bore hole;
a slider block slidable over the cylindrical section;
a fixed handle part attached to the casing at a first end;
a movable handle part attached to the slider block at a first end and joining the fixed handle part;
a sonotrode attached to the converter and running through the central bore hole of the casing;
a covering sleeve covering the sonotrode over a front region;
a push rod attached to the slider block and running inside the covering sleeve;
a clamping jaw hinged to the sonotrode and connected to the push rod for pivoting the clamping jaw hinged to the sonotrode.

3. The surgical instrument according to claim 2 further comprising
a converter receiver supporting the converter;
a flushing line running substantially parallel to the converter receiver and having at one end a connecter for a flushing line leading to a flushing pump;
a suctioning line running substantially parallel to the converter receiver and having at one end a connecter for a suctioning line leading to a suction pump.

4. The surgical instrument according to claim 2 further comprising
an axial channel disposed around the center axis of the sonotrode and inside the sonotrode, wherein the axial channel of the sonotrode is employed as a suction and/or flushing channel.

5. The surgical instrument according to claim 2 wherein the flushing of the application location is performed by a line disposed inside the sonotrode.

6. The surgical instrument according to claim 2 wherein the flushing of the application location is performed by a line disposed parallel to the sonotrode.

7. The surgical instrument according to claim 2 further comprising
a push key for triggering a control of the suction pump or of the flushing pump.

8. The surgical instrument according to claim 7 wherein the push key is arranged in the rigid part of the handle or in the movable part of the handle.

9. The surgical instrument according to claim 2 further comprising
a foot switch connected to the generator or control unit for triggering a control of the suction pump or flushing pump.

10. The surgical instrument according to claim 2 wherein the sonotrode and the covering sleeve are formed exchangeable, and wherein a clamping face of the clamping jaw is furnished with an anti-attachment coating.

11. The surgical instrument according to claim 10 wherein the anti-attachment coating comprises poly tetra fluoro ethylene.

12. The surgical instrument according to claim 2 wherein the fixed part of the handle and the movable part of the handle are connected to each other by a spring made of stainless steel.

13. The surgical instrument according to claim 2 wherein the fixed part of the handle is disposed relatively remote to a tip of the sonotrode, and wherein the movable part of the handle is disposed relatively close to the tip of the sonotrode,

14. The surgical instrument according to claim 13 further comprising
a clamping screw supported by the casing, disposed between the fixed part of the handle and the movable part of the handle and engaging the sonotrode for securing the sonotrode against unintentional rotation, wherein the clamping screw extends in a radial direction relative to an axis of the sonotrode, wherein the clamping screw has a grippable head for manual operation and tightening.

15. The surgical instrument according to claim 13 further comprising
an outer threaded end of the casing disposed adjoining the cylindrical section of the casing;
a swivel nut having an inner thread matching the outer threaded end of the casing and engaging the outer threaded end and having a perforated ring on an end disposed remote to the casing, wherein said perforated ring has an inner diameter larger than an outer diameter of the covering sleeve, wherein the swivel nut has a length longer than a length of the sliding block, and wherein the swivel nut has an outer diameter larger than an outer diameter of the sliding block, and wherein the swivel nut serves as a stop for the sliding block.

16. A method of ultrasonic surgery comprising the steps
attaching a fixed end of a handle to a casing;
attaching a movable end of a handle to a sliding block sliding over a section of the handle;
placing a sonotrode through a central axial bore hole of the casing;
attaching the sonotrode to a converter;
connecting a push rod to the sliding block;
surrounding the push rod and the sonotrode with a covering sleeve;
screwing a swivel nut onto a threaded end of the casing for furnishing a stop to the sliding block.

17. The method of ultrasonic surgery according to claim 16 further comprising the steps:
switching between a cutting and coagulating operation and a dissecting operation.

18. The method of ultrasonic surgery according to claim 17 comprising the step:
pressing a push key for switching between the cutting and coagulating operation and the dissecting operation.
